(19) United States
(12) Patent Application Publication
(10) Pub. No.: US 2007/0235034 A1
(43) Pub. Date: Oct. 11, 2007

(54) HEADBAND FOR HOLDING A NASAL CANNULA ON AN INFANT

(76) Inventor: Deirdre Iris Weaver, Newark, OH (US)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 11/682,046

(22) Filed: Mar. 5, 2007

Related U.S. Application Data
(60) Provisional application No. 60/789,756, filed on Apr. 6, 2006.

(51) Int. Cl. A61M 15/08

(52) U.S. Cl. 128/207.18

(57) ABSTRACT

A device for holding a nasal cannula on a patient includes a band formed of a generally flat strip of material, a first pair of fasteners at a first lateral side of the band for releasably securing one of the medical tubes to the band, and a second pair of fasteners at a second lateral side of the band, opposite the first lateral side, for releasably securing the other of the medical tubes to the band. The first medical tube extends from the nasal cannula to upwardly extend through one of the first pair of fasteners and then loops to downwardly extend through the other of the first pair of fasteners. The second medical tube extends from the nasal cannula to upwardly extend through one of the second pair of fasteners and then loops to downwardly extend through the other of the second pair of fasteners.

### CROSS-REFERENCE TO RELATED APPLICATIONS

This application claims priority from U.S. Provisional Application No. 60/789,756 filed on Apr. 6, 2006, the disclosure of which is expressly incorporated herein in its entirety by reference.

### STATEMENT REGARDING FEDERALLY SPONSORED RESEARCH

Not Applicable

### REFERENCE TO MICROFICHE APPENDIX

Not Applicable

### FIELD OF THE INVENTION

The present invention generally relates to devices for providing breathing assistance and, more particularly, to a headband for holding breathing tubes that lead to a nasal cannula.

### BACKGROUND OF THE INVENTION

The medical profession often uses positive pressure therapy to treat ventalatory insufficiency. Continuous positive airway pressure (CPAP) is typically used when the patient is breathing spontaneously. A nasal cannula is the most typical system used to deliver CPAP to premature and other infants. It is common to secure air or oxygen tubes leading to the nasal cannula to the cheeks of the infant using pressure sensitive adhesive tape. While the adhesive tape may be effective at securing the nasal cannula so that it does not inadvertently slip out of position, it can be very painful to remove the tape such as at bath time. Additionally, continual application and removal of the tape can lead to irritation, sores, and other breakouts that are painful to the infant.

Various head bands and hats have been utilized for securing nasal cannulas and/or other medical tubing to the head of patients. See for example, U.S. Pat. Nos. 4,665,566, 4,739,757, 4,836,200, 5,645,058, 5,704,916, 6,536,436, 6,684,883, 6,899,102, 7,146,976, D468,013, D479,329, and D501,554, the disclosures of which are expressly incorporated herein in their entireties for reference. While these devices may be adequate for securing nasal cannulas to adult patients, they are ineffective for infants and/or small children which have unique requirements.

Accordingly, there is a need in the art for an improved device for holding a nasal cannula on an infant.

### SUMMARY OF THE INVENTION

The present invention provides a device for holding a nasal cannula which overcomes at least some of the problems of the related art. A device for holding a nasal cannula having a pair of medical tubes extending therefrom on a patient comprises, in combination, a band formed of a generally flat strip of material, a first pair of fasteners at a first lateral side of the band for releasably securing one of the medical tubes to the band, and a second pair of fasteners at a second lateral side of the band, opposite the first lateral side, for releasably securing the other of the medical tubes to the band.

According to another aspect of the invention, a nasal cannula and device for holding the nasal cannula on a patient comprise, in combination, a nasal cannula having a first and second medical tubes extending therefrom, a band formed of a generally flat strip of material, a first pair of fasteners at a first lateral side of the band for releasably securing one of the medical tubes to the band, and a second pair of fasteners at a second lateral side of the band, opposite the first lateral side, for releasably securing the other of the medical tubes to the band. The first medical tube extends from the nasal cannula to upwardly extend through one of the first pair of fasteners and then loops to downwardly extend through the other of the first pair of fasteners. The second medical tube extends from the nasal cannula to upwardly extend through one of the second pair of fasteners and then loops to downwardly extend through the other of the second pair of fasteners.

According to yet another aspect of the invention, a method of securing a nasal cannula to a patient comprises, in combination, the steps of providing a nasal cannula having a first and second medical tubes extending therefrom, providing a band formed of a generally flat strip of material, providing a first pair of fasteners at a first lateral side of the band for releasably securing one of the medical tubes to the band, and providing a second pair of fasteners at a second lateral side of the band, opposite the first lateral side, for releasably securing the other of the medical tubes to the band. The method additional includes the step of extending the first medical tube from the nasal cannula to and upwardly through one of the first pair of fasteners and looping the first medical tube downwardly through the other of the first pair of fasteners. The method further includes the step of extending the second medical tube from the nasal cannula to and upwardly through one of the second pair of fasteners and looping the second medical tube downwardly through the other of the second pair of fasteners.

From the foregoing disclosure and the following more detailed description of various preferred embodiments it will be apparent to those skilled in the art that the present invention provides a significant advance in the technology and art of device for holding a nasal cannula. Particularly significant in this regard is the potential the invention affords for providing a high quality, reliable, secure, and easy to use device. Additional features and advantages of various preferred embodiments will be better understood in view of the detailed description provided below.

### BRIEF DESCRIPTION OF THE DRAWINGS

These and further features of the present invention will be apparent with reference to the following description and drawings, wherein:

FIG. 1 is a front elevational view of a device holding a nasal cannula according to the present invention;

FIG. 2 is a left side elevational view of the nasal cannula holding device of FIG. 1;

FIG. 3 a rear elevational view of the nasal cannula holding device of FIGS. 1 to 3;

FIG. 4 is top plan view of the nasal cannula holding device of FIGS. 1 to 3, wherein the nasal cannula has been removed for clarity;

FIG. 5 is a left side elevational view of the nasal cannula holding device of FIGS. 1 to 4 securing a nasal cannula to an infant; and

FIG. 6 is a front elevational view of the nasal cannula holding device of FIGS. 1 to 4 securing a nasal cannula to an infant.

It should be understood that the appended drawings are not necessarily to scale, presenting a somewhat simplified representation of various preferred features illustrative of the basic principles of the invention. The specific design features of holding devices for nasal cannulas as disclosed herein, including, for example, specific dimensions, orientations, locations, and shapes will be determined in part by the particular intended application and use environment. Certain features of the illustrated embodiments have been enlarged or distorted relative to others to facilitate visualization and clear understanding. In particular, thin features may be thickened, for example, for clarity or illustration. All references to direction and position, unless otherwise indicated, refer to the orientation of the control device illustrated in the drawings. In general, up or upward refers to an upward direction within the plane of the paper in FIG. 1 and down or downward refers to a downward direction within the plane of the paper in FIG. 1. Also in general, front or frontward refers to a direction out of the plane of the paper in FIG. 1 and back, rear, or rearward refers to a direction into the plane of the paper in FIG. 1.

### DETAILED DESCRIPTION OF CERTAIN PREFERRED EMBODIMENTS

It will be apparent to those skilled in the art, that is, to those who have knowledge or experience in this area of technology, that many uses and design variations are possible for the device for holding a nasal cannula disclosed herein. The following detailed discussion of various alternative and preferred embodiments will illustrate the general principles of the invention with reference to a preferred embodiment for use with infants. Other embodiments suitable for other applications will be apparent to those skilled in the art given the benefit of this disclosure.

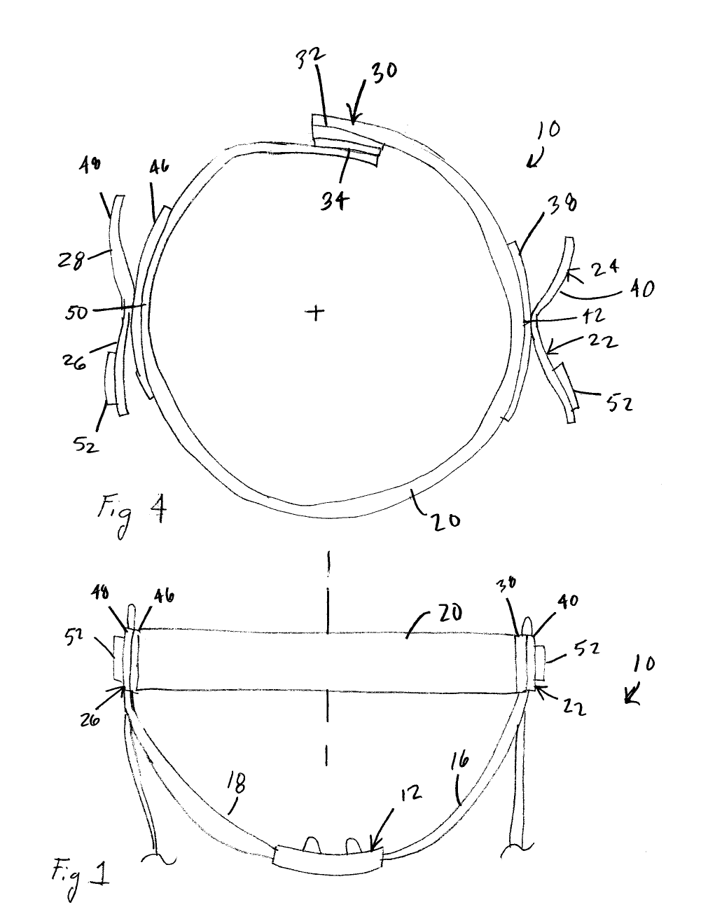

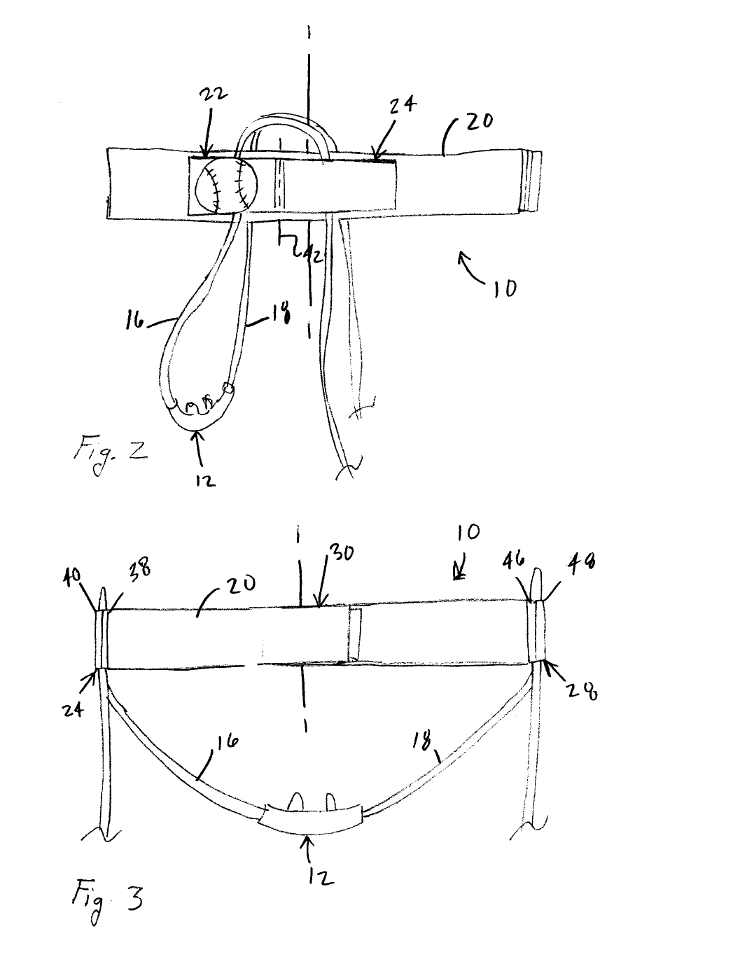

Referring now to the drawings, FIGS. 1 to 4 show a device 10 for holding a nasal cannula 12 on a infant 14 or small child according to the present invention. The illustrated nasal cannula 12 has a pair of medical tubes 16, 18 or the like extending from opposite sides to connect the nasal cannula 12 to a source of oxygen or the like. The illustrated tubes 16, 18 are generally elongate and annular shaped in cross section to form an internal passage for the oxygen from the source to the nasal cannula. The tubes 16, 18 can be formed of any suitable material.

The illustrated device 10 includes a band 20 formed of a generally flat strip of material for extending about the patient's head, a first pair of fasteners 22, 24 at a first lateral side of the band 20 for releasably securing one of the medical tubes 16, 18 to the band 20 and, a second pair of fasteners 26, 28 at a second lateral side of the band 20, opposite the first lateral side, for releasably securing the other of the medical tubes 16, 18 to the band 20.

The illustrated band 20 is sized and shaped to extend around the head of the infant 14. The illustrated band 20 is generally elongate and rectangular-shaped when in its open full and flat length position. The length and width of the band 20 can be of a variety of different suitable dimensions to accommodate different size infants. The illustrated band 20 has a length of about 17 inches, a width of about 1 inch, and a thickness of about 1/16 of an inch. The band 20 preferably at least partially comprises an elastic material but any suitable material can be utilized.

The illustrated band 20 is provided with a releasable fastener 30 for releasably securing the band about the infant's head and adjusting the size of the band 20. The releasable fastener 30 can be of any suitable type such as, for example, hook-and-loop, buttons, snaps, hooks, or any other suitable fastener. The illustrated releasable fastener 30 is a hook and loop fastener wherein a loop portion 32 is secured to one end of the band 20 at an inner side and a hook portion 34 is secured to the other end of the band 20 at an outer side. It is noted that the hook and loop portions 32, 34 can be reversed if desired. The illustrated hook-and-loop releasable fastener 30 is secured to the band 20 by sewn stitches but any other suitable securing means can alternatively be utilized.

The first pair of fasteners 22, 24 are provided at a first lateral side of the band 20 for releasably securing the first medical tube 16 to the band 20 generally above an ear 36 of the infant 14. The first pair of releasable fasteners 22, 24 can be of any suitable type such as, for example, hook-and-loop, buttons, snaps, hooks, or any other suitable fastener. The illustrated first pair of releasable fasteners 22, 24 are each hook and loop fasteners. Each fastener 22, 24 is formed by securing a strip of hook portion 38 along the length of the band 20 on the outer side and securing a strip of loop portion 40 only at the center of the hook portion 38 so that the loop portion 40 is facing the hook portion 38 and forms a tab bendable about the connection which forms a generally vertical pivot axis 42 for the tab. It is noted that the position of the hook portion 38 and the loop portion 40 can be reversed if desired. Thus, the illustrated tabs or loop portions 40 open and close in opposite directions to form the pair of adjacent fasteners 22, 24. The illustrated first pair of hook-and-loop fasteners 22, 24 are secured to the band 20 by sewn stitches but any other suitable securing means can alternatively be utilized. The illustrated first pair of fasteners 22, 24 are formed by a single strip of hook material 38 and a single strip of loop material 40 so that the separate fasteners are contiguous at the central attachment of the loop portion 40. By extending along the length of the band 20, the fasteners 22, 24 permit the first medical tube 16 to be secured to the band 20 in vertical orientations as described in more detail hereinafter.

The second pair of fasteners 26, 28 are provided at a second lateral side of the band 20 for releasably securing the second medical tube 18 to the band 20 generally above the other ear 44 of the infant 14. The second pair of releasable fasteners 26, 28 can be of any suitable type such as, for example, hook-and-loop, buttons, snaps, hooks, or any other suitable fastener. The illustrated second pair of releasable fasteners 26, 28 are hook and loop fasteners. Each fastener 26, 28 is formed by securing a strip of hook portion 46 along the length of the band on the outer side and securing a strip of loop portion 48 only at the center of the hook portion 46 so that the loop portion 48 is facing the hook portion 46 and forms a tab bendable about the connection which forms a generally vertical pivot axis 50. It is noted that the position of the hook portion 46 and the loop portion 48 can be reversed if desired. Thus, the tabs or loop portions 48 open and close in opposite directions to form the pair of adjacent fasteners 26, 28. The illustrated second pair of hook-and-loop fasteners 26, 28 are secured to the band 20 by sewn stitches but any other suitable securing means can alternatively be utilized. The illustrated second pair of fasteners 26, 28 are formed by a single strip of hook material 46 and a single strip of loop material 48 so that the separate fasteners 26, 28 are contiguous at the central attachment of the loop portion 48. By extending along the length of the band 20, the fasteners 26, 28 permit the second medical tube 18 to be secured to the band 20 in vertical orientations as described in more detail hereinafter.

The forward outer ends of the illustrated front fasteners 22, 26 are each provided with decorative indicia 52. The illustrated indicia 52 is in the form of a baseball but the indicia can be of any form that makes the device more desirable to where by the infant or small child 14. The indicia 52 can be formed of any suitable material and can be secured in any suitable manner. It is noted that any suitable quantity of the indicia 52 can be provided and the indicia 52 can be provided at any suitable location.

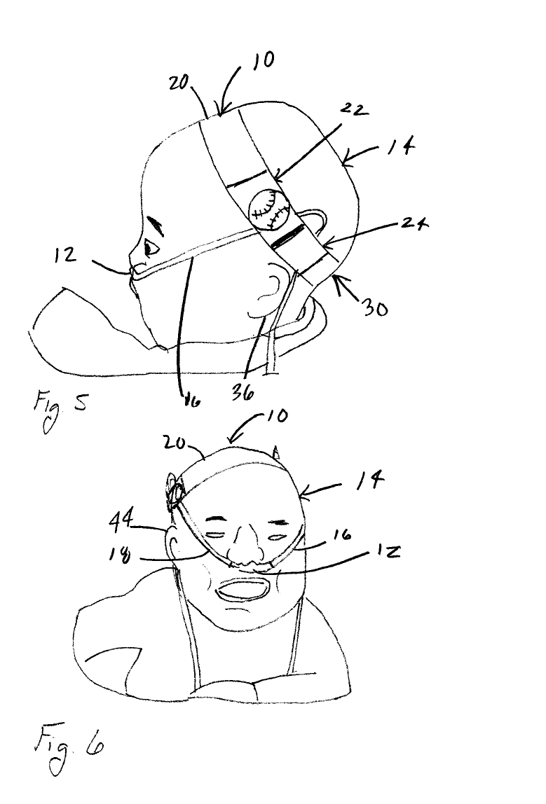

As shown in FIGS. 5 and 6, the band 20 is placed about the infants head and the releasable fastener 30 is closed to secure the band 20 about the infants head. The illustrated fastener 30 enables the band to be adjustable for different sizes of infant heads. The illustrated band 20 is adjustable to fit heads of about 16½ inches to about 12 inches in circumference but any other suitable sizes can be alternatively utilized. The nasal cannula 12 is placed in the infant's nose and the medical tubes 16, 18 extend laterally from the nasal cannula 12 in opposite directions. Each medical tube 16 18 extends upwardly at the side of the infants head and upwardly passes through the front fastener 22, 26 and then loops to pass downwardly through the rear fastener 24, 28 and then extends downwardly to the source of oxygen.

It is apparent from the forgoing that the present invention provides an improved holding device in that the front and rear fasteners each enable the air tubes to be held in a secure manner at an infinite number of positions along the side of the infants head by simply placing the medical tubes in a desired position and closing the hook-and-loop fastener over the air tube. Thus, the holding device is able to be easily used on differing sized infant heads to secure the medical tubes in a desired position.

From the foregoing disclosure and detailed description of certain preferred embodiments, it is also apparent that various modifications, additions and other alternative embodiments are possible without departing from the true scope and spirit of the present invention. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

What is claimed is:

1. A device for holding a nasal cannula having a pair of medical tubes extending therefrom on a patient, said device comprising, in combination:
a band formed of a generally flat strip of material;
a first pair of fasteners at a first lateral side of the band for releasably securing one of the medical tubes to the band; and
a second pair of fasteners at a second lateral side of the band, opposite the first lateral side, for releasably securing the other of the medical tubes to the band.

2. The device according to claim 1, wherein the first pair of fasteners and the second pair of fasteners each comprise hook and loop fasteners.

3. The device according to claim 1, wherein the first pair of fasteners are adjacent one another and the second pair of fasteners are adjacent one another.

4. The device according to claim 3, wherein the first pair of fasteners are contiguous and the second pair of fasteners are contiguous.

5. The device according to claim 1, wherein the first pair of fasteners open in opposite directions and the second pair of fasteners open in opposite directions.

6. The device according to claim 1, wherein the first pair of fasteners and the second pair of fasteners each secure the medical tubes in a vertical orientation.

7. The device according to claim 1, wherein at least one of the fasteners is provided with decorative indicia thereon.

8. The device according to claim 1, wherein the first and second pairs are fasteners are positioned to be located generally above ears of the patient.

9. A nasal cannula and device for holding the nasal cannula on a patient comprising, in combination:
a nasal cannula having a first and second medical tubes extending therefrom;
a band formed of a generally flat strip of material;
a first pair of fasteners at a first lateral side of the band for releasably securing one of the medical tubes to the band;
a second pair of fasteners at a second lateral side of the band, opposite the first lateral side, for releasably securing the other of the medical tubes to the band;
wherein the first medical tube extends from the nasal cannula to upwardly extend through one of the first pair of fasteners and then loops to downwardly extend through the other of the first pair of fasteners; and
wherein the second medical tube extends from the nasal cannula to upwardly extend through one of the second pair of fasteners and then loops to downwardly extend through the other of the second pair of fasteners.

10. The device according to claim 9, wherein the first pair of fasteners and the second pair of fasteners each comprise hook and loop fasteners.

11. The device according to claim 9, wherein the first pair of fasteners are adjacent one another and the second pair of fasteners are adjacent one another.

12. The device according to claim 11, wherein the first pair of fasteners are contiguous and the second pair of fasteners are contiguous.

13. The device according to claim 9, wherein the first pair of fasteners open in opposite directions and the second pair of fasteners open in opposite directions.

14. The device according to claim 9, wherein the first pair of fasteners and the second pair of fasteners each secure the medical tubes in a vertical orientation.

15. The device according to claim 9, wherein at least one of the fasteners is provided with decorative indicia thereon.

16. The device according to claim 9, wherein the first and second pairs are fasteners are positioned to be located generally above ears of the patient.

17. A method of securing a nasal cannula to a patient comprising, in combination, the steps of:
providing a nasal cannula having a first and second medical tubes extending therefrom;
providing a band formed of a generally flat strip of material;
providing a first pair of fasteners at a first lateral side of the band for releasably securing one of the medical tubes to the band;
providing a second pair of fasteners at a second lateral side of the band, opposite the first lateral side, for releasably securing the other of the medical tubes to the band;
extending the first medical tube from the nasal cannula to and upwardly through one of the first pair of fasteners and looping the first medical tube downwardly through the other of the first pair of fasteners; and
extending the second medical tube from the nasal cannula to and upwardly through one of the second pair of fasteners and looping the second medical tube downwardly through the other of the second pair of fasteners.

18. The method according to claim 17, further comprising the step of providing hook and loop fasteners for each of the first pair of fasteners and the second pair of fasteners.

19. The method according to claim 17, further comprising the step of providing at least one of the fasteners with decorative indicia thereon.

20. The device according to claim 9, further comprising the step of positioning each of the first and second pairs to be located generally above ears of the patient.
